(19) United States
(12) Patent Application Publication
(10) Pub. No.: US 2010/0291223 A1
(43) Pub. Date: Nov. 18, 2010

(54) TREATMENT FOR DERMATOLOGICAL CONDITIONS

(76) Inventor: Robert Alan Armstrong, Kingmeadows (AU)

Correspondence Address: Pearne & Gordon LLP, 1801 East 9th Street, Suite 1200, Cleveland, OH, 44114-3108, US

(21) Appl. No.: 12/812,002

(22) PCT Filed: Jan. 6, 2009
(86) PCT No.: PCT/AU09/00012

§ 371 Date: Aug. 3, 2010

(30) Foreign Application Priority Data
Jan. 7, 2008 (AU) 2008900052
May 23, 2008 (AU) 2008902587

(51) Int. Cl. A61K 9/14; A61K 35/60; A61K 36/61

(52) U.S. Cl. 424/489; 424/523; 424/725; 977/773

(57) ABSTRACT

A composition and method of using a composition containing kunzea oil for treating dermatological conditions in humans and animals, particularly greasy heel occurring in horses, in which the kunzea oil is formulated into a composition for topical application at ambient temperatures to the skin of the animal or human to cover the area of the affected lesions. Improvement in certain dermatological conditions such as pastern dermatitis in horses was noted almost immediately with total resolution of the condition in about 7 days. The composition can be used to treat humans suffering from psoriasis and similar dermatological conditions. This invention offers persons and animals suffering from dermatological conditions a low cost effective treatment based on a natural product which shows significant improvement of the condition within days of applying an effective amount of the composition to the affected area as part of the treatment.

### FIELD OF THE INVENTION

The present invention relates to compositions for use in the treatment of dermatological conditions.

Additionally, the invention relates to methods of using such compositions to treat dermatological conditions.

In one form the invention relates to compositions and methods for the treatment of dermatological conditions in humans.

In one form the invention relates to compositions and methods for the treatment of dermatological conditions in animals that suffer from dermatological ailments, and, in particular, animals with unpigmented skin which are susceptible to developing conditions on exposure to the elements, such as water, sun, heat or the like.

In one aspect the present invention finds particular application to compositions and methods for the treatment of dermatitis occurring within the legs of horses, particularly dermatological conditions known as “greasy heel” or similar conditions including, but not exclusively, solar keratosis in cats, dogs, horses, alpacas, and particularly on ears and noses of animals with unpigmented skin and white points.

In one form the present invention finds particular application to compositions and methods for treatments of skin conditions in humans, such as for example psoriasis, onychomycosis, and related skin conditions.

Although the present invention will be described with particular reference to the treatment of the dermatological condition of greasy heel in horses, it is to be noted that the present invention also includes within its scope the treatment of dermatological conditions in humans and a wide range of animals and is not restricted to treating horses. Further, although the invention is described with particular reference to treating pastern dermatitis in horses, the scope of the invention is not restricted to treating this condition but includes treating many other dermatological conditions.

### BACKGROUND OF THE INVENTION

Pastern dermatitis, also known as grease or greasy heel, scratches, mauke or mud fever, is a progressive dermatitis. Usually, this condition begins with erythematous lesions in the plantar pastern region, more often of the hind legs. Lesions typically develop scaling, that progresses to the release of a malodorous exudate and crust formation, with associated alopecia. Oedema is common and may extend to the fetlock and lower cannon area. Lesions may spread to the dorsal pastern and cannon regions. Due to constant flexion, skin fissures may develop from which, in severe cases, bleeding is common. With chronicity, more often in heavy breeds, nodular skin masses or verrucous skin lesions may develop. Acute lamenedd can result from severe cases when treatment is neglected or is unsuccessful.

Pastern dermatitis is a syndrome that may involve various inflammatory skin conditions that arise from a number of different pathophysiological processes. These include mite infestation, bacterial infection, dermatophytosis, dermatophilosis, photosensitisation, vasculitis, vaccinia, pemphigus foliaceus and primary irritant contact dermatitis.

In the past there has been little or no scientific clinical studies conducted that have thoroughly evaluated treatment options for pastern dermatitis. Anecdotal reports suggest that treatment is often initiated by horse owners and that various home remedies are used, including preparations containing substances such as copper sulphate, kerosene and sulphur. Not surprisingly, the condition often persists for months or even years since such treatments are largely ineffective, having little or no scientific basis.

Treatment by veterinarians is commonly empirical and is determined by the stage of the disease, often with little or no improvement caused by the treatment, just a general improvement in the health of the animal due to more attention and care to the animal, again because there is little or no scientific knowledge about treatment of this condition. Treatment and preventative recommendations include separation of the horse from potential agents of the disease by its removal from wet, muddy and otherwise unhygienic conditions, as well as avoidance of potential chemical, plant-derived or environmental irritants and similar. However, to date there has not been any treatment of pastern dermatitis that has been wholly satisfactory or effective. The same can be said of other related conditions in both humans and animals.

Previously available treatments of pastern dermatitis are either ineffective, or at best are only marginally effective with only slight improvement being observed and then only after a prolonged period of treatment. Thus, there is a need for a simple, clinically-proven treatment option for non-severe cases of pastern dermatitis that has greater effectiveness than existing treatments, and preferably showing an improvement in the condition occurring within a shorter time.

Accordingly, it is one aim of the present invention to provide a composition for the treatment for pastern dermatitis in horses which is at least as effective as existing treatments for this condition.

Accordingly, it is one aim of the present invention to provide a method of using a composition to treat pastern dermatitis in horses.

Accordingly, it is one aim of the present invention to provide a method and composition for the effective treatment of a dermatological condition in horses known as greasy heal.

Accordingly, it is one aim of the present invention to provide a composition and method for the treatment for dermatological conditions in humans.

Accordingly, it is one aim of the present invention to provide a composition and method for the treatment of any one or more of psoriasis, onychomycosis, dermatitis, or similar.

It is to be noted that not all embodiments of the present invention will satisfy all aims of the invention. One embodiment will satisfy one aim whilst another embodiment will satisfy another aim. Some embodiments will satisfy two or more aims.

One problem of existing formulations for use in dermatological treatments relates to topical application of creams and pastes containing an active ingredient. However, such previously available creams, pastes and the like can only be applied topically within certain temperature ranges such as for example below a certain temperature. In the past existing preparations could only be used below a threshold temperature since one of the components of such compositions, Kunzea oil, an essential oil from the shrub Kunzea ambigua, is liquid at ambient temperatures so that compositions containing this essential oil were suitable for use only at relatively low ambient temperatures because the viscosity of the composition containing this oil is strongly temperature dependent, i.e. the viscosity reduces as the temperature increases until the viscosity is too low to allow topical application because the composition will not adhere to the affected area to which it has been applied. Accordingly, when the composition warms up, such as shortly after contact with the warm skin of an animal, on exposure to higher ambient air temperatures, to sunlight and similar, the viscosity of the composition is reduced to an extent that the composition cannot be applied in an effective manner to cover the area being treated so as to be retained on the treated area, but rather its use was reduced to an extent that the composition simply runs off the area to which the composition is topically applied without adhering to the affected area. The effect of the composition in treating the dermatological condition is, therefore, largely ineffective owing to the poor coverage and duration of the treatment. Thus, there is a need for a composition that is effective when applied topically over a wider range of temperatures, particularly when applied at temperatures within a certain threshold temperature range, including temperatures at which the viscosity of compositions are conventionally reduced to an extent that the composition is ineffective.

Thus, it is an aim of the present invention to provide an improved treatment for dermatological conditions in animals, including humans, that can be applied over a wider range of temperatures, particularly temperatures, above ambient temperatures than can existing treatments being applied topically.

### SUMMARY OF THE INVENTION

According to one aspect of the present invention there is provided composition for the treatment of a dermatological condition, said composition comprising a kunzea oil and/or a myrtaceous oil or a precursor of kunzea oil and/or myrtaceous oil, or a derivative of kunzea oil and/or myrtaceous oil, said composition being suitable for topical application to an area of skin that is affected by the dermatological condition over a range of temperatures, and wherein the viscosity modifier is incorporated into the composition for reducing temperature dependence of the viscosity of the composition to facilitate topical application of the composition to the affected area.

According to another aspect of the present invention there is provided a method of treating a dermatological condition comprising the steps of applying an effective amount of a therapeutic composition to a part of the body having the dermatological condition wherein the composition comprises a kunzea oil and/or a myrtaceous oil or a precursor of kunzea oil and/or myrtaceous oil, or a derivative of kunzea oil and/or myrtaceous oil and a viscosity modifier, said composition being suitable for topical application within a predetermined temperature range and wherein the viscosity modifier is provided for reducing temperature dependence of the viscosity of the composition thereby facilitating topical application of the composition to the affected area so that the composition remains where applied to a greater extent to provide an effective treatment.

Preferably, the composition further includes an additive or auxiliary material. Typically, the additive or auxiliary material is salicylic acid, sulphur powder, cod liver oil, a homogenizing agent or the like. Typically, the homogenizing agent is any suitable homogenising agent. Other materials that can be added to the composition include one or more of the following either alone, or in combination:

Triethanolmanine is an emulsifying and stabilising agent which allows the incorporation of the oils in to the composition in the form of an ointment.

Zinc oxide is used as a potent sun screen agent and viscosity enhancer.

Kunzea oil is an antibacterial, antifungal and anti-inflammatory natural oil.

The composition may include coal tar solution.

Preferably, the viscosity modifier causes the composition to form an ointment, cream, lotion or similar, suitable for topical application for temperatures up to about 50° C., preferably within the range 0° C. to 45° C., more preferably within the range from about 5° C. to about 45° C., most preferably, from about 10° C. to 45° C.

Preferably, the viscosity modifier is zinc oxide.

Typically, the zinc oxide is in the form of a powder. More typically, in one form, the particle size of the powder is from about 0.01 mm to about 0.002 mm. More typically, the powdered zinc oxide can be micron sized powder, such as for example, having a particle size of greater than or about equal to 100 nm, or be nano-sized powder, such as, submicron nanometre-sized high purity zinc oxide having a particle size less than 1 μm.

In one form, the zinc oxide powder is in the form of aggregates. More typically, the aggregates include particles of different morphologies which include circular, ellipsoidal, linear or branched forms. In one form, the powdered zinc oxide is in the form of aggregates composed of particles having different morphologies in which about 0 to 10% of the aggregates are in circular form, 30 to 50% of the aggregates are in the ellipsoidal form, 30 to 50% of the aggregates are in the linear form, and 20 to 30% of the aggregates are in the branched form.

In one form, the particle size of the powdered zinc oxide is from about 0.01μ to about 200μ, preferably from about 0.1μ to about 100μ, more preferably from about 0.12μ to about 5μ, and most preferably about 0.12μ to about 2.2μ. Typically, more than 99.9% of the zinc oxide particles pass through a mesh size of −325. However, other particle size distributions are possible including—100 mesh, 10-50 microns, submicron (<1 micron), or the like. In one form, the zinc oxide has extremely narrow particle size distribution.

In one form, the composition is in the form of a low to medium viscosity lotion. However, the composition can take other forms than the lotion, such as being in the form of creams, gels, ointments or other high viscosity composition. A lotion form is preferred as the composition may be removed by shampooing or similar.

Typically, the composition in accordance with the present invention has a higher viscosity at a given temperature than similar compositions with zinc oxide for two reasons.

First, zinc oxide, when added as a powder to form the composition of the invention, increases the viscosity of the composition, thereby making the composition more amenable to use as a topical application. Although the composition will still absorb heat from the skin of the animal to which the composition is applied, the zinc oxide increases the viscosity to an extent that the composition remains in the form of a topical lotion or cream at body temperatures of animals and thus remains adhered to the skin thereby providing more prolonged and effective treatment.

Second, greasy heel and similar conditions are caused by solar influence and an agent is needed to block the solar affect on animals particularly those with unpigmented skin and white points. The zinc oxide in the composition assists in screening out the ultra violet light. The zinc oxide in the composition reduces heat being absorbed into the composition thereby reducing the tendency for the viscosity of the composition to be reduced to an extent that the composition flows or runs off the affected area by the composition liquefying. Accordingly, the addition of viscosity modifier, particularly the zinc oxide, to the composition of the invention results in the composition having improved properties, including improved viscosity, for topical application to the legs of horses in circumstances where the composition is exposed to sunlight and/or hot ambient temperatures, such as for example, when treating horses in the open, in fields, paddocks, stables or the like which is the usual place where horses suffering from this condition are treated, particularly in countries having a “hot” climate, such as a Mediterranean climate or the like, including parts of Australia, U.S.A. and similar.

According to another aspect of the present invention there is provided a method of treating a dermatological condition in an animal, including a human animal, comprising applying an effective amount of a therapeutic composition to an area affected by the dermatological condition, in which the composition comprises an essential oil obtained from a suitable shrub containing a suitable therapeutic oil and a viscosity modifier.

It is to be noted that the therapeutic oil useful in the present invention includes all forms of kunzea oil obtained from all kunzea species, and all myrtaceous oils that have a similar chemical composition to that of the oil from Kunzea ambigua and/or all myrtaceous oils that have a similar profile of antifungal and antibacterial activity as demonstrated by the oil from Kunzea ambigua. Examples of myrtaceous oils include: oils from Kunzea spp. including K. baxteri, K. ericoides, K. pomifera and K. ericifolia; oils from Melaleuca spp. including M. alternifolia and M. quinquinervia; oils from Leptospermum spp. including L. scoparium and L. petersonii; oils from Syzygium spp, including Z. aromaticum and Z. gardneri.

Preferably the essential oil derived from Kunzea ambigua is Kunzea oil, and more preferably the Kunzea oil contains as major components α-pinene, 1,8-cineole, α-terpineol, bicyclogermacrene, δ-cadinene, citronellol, calamenene, ledol, globulol and/or, viridflorol. The composition can be applied to the affected area in any amount, and any dosage and any number of times.

Preferably, the composition of the present invention is applied to the area of skin that is affected by the dermatological condition twice a day to the affected area of skin.

Preferably, the composition and the treatment are suitable for treating psoriasis, seborrheic dermatitis, solar keratosis, fungal infections, greasy heel, dermatitis, psoriasis, onychomuycosis, dermatitis, or the like.

Preferably, the composition and the treatment are suitable for treating dermatological conditions in horses, cats, dogs, alpacas and similar animals, more preferably on the legs, ears, noses of animals with unpigmented skin and white points. Preferably, animals with unpigmented skin are treatable for dermatological conditions using the composition and method of the present invention.

### DETAILED DESCRIPTION OF THE INVENTION

In one embodiment kunzea oil is obtained by steam distillation of the herb Kunzea ambigua. Kunzea oil is pale yellow in colour and is a liquid at 20° C. and has a relative density of 0.912 at that temperature. The active ingredient in kunzea oil for treating skin conditions is mainly the sesquiterpenes. However other components of Kunzea oil can contribute to treating skin conditions either alone or in combination with the sesquiterpenes.

Compositions including kunzea oil in the range of 10 to 40 weight % based on the total weight of the composition provide effective relief to animals suffering from skin conditions such as seborrheic dermatitis, solar keratosis, fungal complaints, or similar.

Salicylic acid BP is added to the composition as a keratolytic agent to aid in treating the skin conditions due to fungal infections since this material is an effective antifungal agent by softening the outer horny layer of skin allowing skin shedding.

Sulphur powder BP is added to the composition as a keratolytic agent since this material is an antiseptic, particularly when combined with salicyclic acid.

In one form, a mixture of salicylic acid and sulphur is used.

Cod liver oil BP is added to the composition as a healing agent to promote quicker healing of the lesion, and to act as a moisturiser.

Triethanolamine BP 10% is added to the composition as a stabilizing agent to enhance the emulsifying ability of the oils and ointment to provide the production of a more homogenous mixture of ingredients or components.

Emulsifying ointment BP is added to the composition to provide the base to enhance and to form a more or less homogenous mixture of the various components.

It is believed that one or more of the above ingredients may be substituted with functionally similar ingredients to provide the same effect and such substituents fall within the scope of the present invention.

Testing carried out by the School of Pharmacy at the University of Tasmania verifies the optimal temperature range for the composition is 10° C. to 45° C.

Examples of the present invention will now be described to illustrate aspects of the invention.

### Example 1

The purpose of this study was to evaluate the efficiency of an ointment formulation containing kunzea oil in the treatment of pastern dermatitis in horses, within a randomised controlled design with the result that as verified by studies conducted by the School of Pharmacy at the University of Tasmania, the formulation was found to be very effective.

Horses from either gender, with localised pastern dermatitis and without any other obvious signs of disease or injury, were selected to take part in this study. The samples of Kunzea oil used in the test procedure were obtained from J. J. Hood, Waterhouse, Tasmania. Ketoconazole, the active component corresponding to Kunzea oil used in the control samples of this study was obtained from Sharon Pharmaceuticals (Mumbai, India). The test and control ointments were prepared following Australian Pharmaceutical Formulary and Handbook guidelines for ointment preparation, at the School of Pharmacy, University of Tasmania. Each formulation contained pharmaceutical grade zinc oxide (5 percent w/w), salicyclic acid (5 percent w/w), precipitated sulphur (5 percent w/w), triethanolamine (1 percent v/w), and cod liver oil (10 percent v/w). The control formulation contained ketoconazole (2 percent w/w) and the test formulation contained kunzea oil (20 percent v/w). Emulsifying ointment was added to make each sample up to 100 percent. The same emulsifying ointment was used in all cases.

The treatment of each horse, after the initial assessment, involved cleaning and drying of the affected pasterns without the use of antiseptic solutions. Crusts were removed. In a few cases, the horse required sedation for this process to be possible. Surgical scissors were used to remove hairs from around each lesion. The ointment was then applied liberally, by a veterinarian. The treatment was continued by the horse carer until the next assessment after having been introduced to the proper care of the horses by the veterinarians.

### Assessment

The primary outcome measure was the total lesion area, which was the sum of the areas of all the pastern dermatitis lesions on an animal. Where a horse had more than one lesion, the largest lesion was termed the primary lesion. The lesion areas were measured by a method similar to the acetate tracing technique. A sterile adhesive transparent sheet was placed over the wound surface and the lesion perimeter was traced using a fine-tipped permanent marker pen. The tracings were subsequently photocopied onto paper and the paper was cut along the line that defined the lesion border. The resultant paper cutout was weighed (to 0.1 mg) and the area corresponding to the lesion area, was determined from the unit area mass of the paper.

Seven clinical signs associated with pastern dermatitis were identified. Each affected pastern being treated in this trial was assessed for exudate, crusting, skin fissures, alopecia, skin excoriation, hypo-pigmentation and weals. A 0-4 linear visual analogue scale in which 0=none; 1=some; 2=mild; 3=moderate and 4=severe was used to identify and record the affected areas. Scores were added to yield a “lesion score” which had a possible maximum value of 28 per pastern. A total lesion score was obtained by adding together the lesion scores from each pastern from the individual scores of the above indicated seven assessments. This was used as a secondary outcome measure.

One lesion, termed the target lesion, was selected by the veterinarian for taking pathology specimens. This was usually, but not always, the largest lesion present. A tissue sample (crust and/or hairmat) was taken and sent to be cultured for Staphylococcus spp., Dermatophilus congolensis, Microsporum spp., Trichophyton spp., and Malassezia pachydermatis. Exudate from the lesion was smeared onto two sterile glass slides that were sent for examination. All pathological testing was performed by Idexx Laboratories (Brisbane, Australia), a certified veterinary pathology laboratory, using standard methods. Cultures were grown on commercially prepared selective media. Smears were Gram-stained prior to microscopic examination.

The initial assessment was performed before any treatment. Follow-up assessment was performed after 7 days treatment. Lesions were measured and scored as described above. In cases of complete resolution of pastern dermatitis signs, a skin scraping and hair sample were taken from the target lesion for microbial culture and contact smears on sterile glass slides (×2) were obtained for microscopic examination. In cases that did not improve after 7 days the treatment medication was switched to the alternative medication (crossover). In cases that improved but did not resolve after 7 days, treatment was continued using the allocated medication for up to 28 days. A further follow-up assessment was carried out after 28 days or earlier if complete resolution occurred. Lesions were measured and scored as before and samples were obtained for pathological testing, as described above.

### Statistical Analysis of Results

All statistical analyses were performed using StateView 5.0 (SAS Institute Inc., Cary, N.C., USA). For comparisons within test or control groups the Wilcoxon signed rank test was used. For comparison of values between the test and control groups the Mann-Whitney test was employed. The frequencies of distribution of the positive pathology results were analysed using the Chi-square test. For all analyses, values of P<0.05 were considered significant. All the data were reported as median (and range) or mean (±sd).

### Results

### Study Subjects

Thirty seven horses were recruited for this trial as shown in Table 1, of which 21 completed the study. The recruitments included 24 females and 13 males represented by 13 thoroughbreds, 10 Arabians, 1 American quarter, 3 ponies, 4 thoroughbred cross breeds, 4 Arabian-cross breeds and 2 Warmbloods. The median duration of disease prior to enrolment was 7 months. Baseline demographic and morphological features of the study population were statistically analysed and no relationships were found between disease severity (total lesion area) and age, gender, pastern colour, breed, disease duration or housing.

Among the 37 horses enrolled, 16 did not undergo an evaluation after 7 days treatment. A major reason for the high dropout rate was the reluctance of owners to pay for a veterinary visit after 7 days, which accounted for 6 horses. Two owners suffered a horse-inflicted injury and did not continue with the study, which accounted for the dropout of 7 horses. In one case the veterinarian was unable to attend 2 horses for their 7-day assessment. One horse was rejected from the study because of a deviation from the study protocol. A total of 21 horses underwent 7 days of treatment with an assessment after this time period. In both test and control groups a comparable mix of horse breeds was present. There were no statistically significant differences between the dropouts and horses completing the trial, or between the two groups of horses completing treatment, in terms of gender, breed, housing conditions, pastern colour, pastern affected or duration of condition.

### Assessment

The results of the Day 0 and Day 7 assessments are summarised in Table 2. Lesions areas of the test and control groups were not different on Day 0 (P>0.05, Mann-Whitney test). On Day 7, the control lesion area had not changed significantly. The lesion areas of the test group had decreased significantly (P<0.01) from a total lesion area of median value 40 cm² (range 3-252 cm²) to 0 cm² (range 0-34 cm²). The response of the test group was significantly greater than the response of the control group (P<0.01, Wilcoxon signed-rank test), thus demonstrating the beneficial results obtained using the composition and method of the present invention for treating horses with this skin condition.

Lesion scores of test and control groups were no different on Day 0. On Day 7, the lesion scores of the control group had not changed significantly, however the lesion scores of the test group had decreased significantly (P<0.05, Mann-Whitney test) from a total lesion score median value of 7 (range 3-36) to a median value of 1 (range 0-9). The response of the test group was significantly greater than the response of the control group (P<0.01, Wilcoxon signed-rank test), providing further evidence for the efficacy of the composition and method of the present invention.

Of the 11 horses treated with test ointment, after 7 days 7 were fully resolved with 100% decrease in lesion area and 4 had improved, with a mean decrease in total lesion area of 71±29%. Of the 10 horses treated with the control ointment, after 7 days 2 were fully resolved, 2 showed some improvement (with a mean decrease in total lesion area of 23±6%) and continued with the same treatment, and 6 showed no improvement or their condition worsened. Of the 6 horses whose condition did not improve, 4 underwent crossover to the test ointment and one was lost to further follow-up.

Of the 2 horses that continued on control ointment, neither was fully resolved after 28 days. One of these horses underwent crossover to the test ointment. Of the total 6 horses that crossed over to the test ointment 5 were fully resolved within 28 days (mean time to healing was 14 days). The single treatment failure experienced an injury to the affected pastern and could not continue with treatment.

The 4 horses from the test group that did not fully resolve within 7 days continued treatment for a further 21 days. After a total of 28 days, two horses were fully healed and the other two (both from the same premises) had marginal improvement. One of these had 4 legs affected. Lesions fully healed on one leg after 7 days, and on another two legs after 28 days, leaving one lesion unhealed. This lesion appeared to be resistant to treatment, having no decrease in area (from 11 cm² to 13 cm²) over four weeks. No further investigation was performed on this lesion.

The pathology results are summarised in Table 3. The most commonly isolated microorganism was Staphylococcus aureus, which was present in 75% of lesion sites before the commencement of treatment, based on culture results. Fungi were cultured from lesion sites in only a few cases. The presence of S. aureus was similar in both control and test groups at Day 0. Follow-up pathology samples were obtained either upon full healing (lesion area reduced to 0 cm²), before crossover (when treatment did not reduce lesion area) or after 28 days of treatment (when treatment reduced lesion area but not to 0 cm²). In three control cases follow-up pathology samples could not be obtained. After the treatment period there was a significant decrease in the presence of cultured S. aureus in the test group compared with the control group (P<0.05, Chi-square test).

Adverse events were not observed by the owners or treating veterinarians for either test or control formulations.

### Discussion

The effects of pastern dermatitis can range from inconsequential to seriously debilitating.

The study of the present example assessed the efficacy of an ointment containing kunzea oil in the treatment of pastern dermatitis. There were several options for the control medication used in the study. A clinically proven standard pastern dermatitis treatment would have been the control of choice, but there does not appear to be such a product available, at least on the Australian market and thus there is no directly comparable treatment currently available to that of the present invention. A placebo treatment was not considered appropriate on ethical grounds. An appropriate control treatment would have been the test ointment minus the kunzea oil component (vehicle only), since the ointment base contains excipients that would be expected to have some potential benefit, such as zinc oxide, salicyclic acid and sulphur. However, at the time of study design the potent antifungal activity of kunzea oil had been recognised by the experimenters and, given the potential involvement of fungi in pastern dermatitis, it was decided to include an antifungal agent in the control ointment. It was also considered, on ethical grounds, more appropriate to offer horse owners a control ointment with a greater potential for activity than a vehicle-only control. Ketoconazole (2%), which was included in the control formulation, has potent antifungal activity in topical applications. In addition ketoconazole has some anti-inflammatory and antibacterial activity. This choice of control ointment allowed the relative importance of the purported major active ingredient of the test ointment, kunzea oil, to be determined. Also some differentiation of the relative importance of its antifungal activity versus its antibacterial activity, in the treatment of pastern dermatitis, could be ascertained.

The results of the study clearly demonstrated the efficacy of the kunzea oil ointment in the treatment of pastern dermatitis in this particular cohort of horses. More than half the cases were completely cured and all cases improved, on the basis of clinical signs and lesion areas, after one week of treatment. Reports from horse owners indicated than in some cases lesions improved dramatically within a few days. By comparison, only two of the horses with the control formulation were cured after one week of treatment, two improved and six did not. Statistically, the kunzea oil formulation was significantly more effective than control, on the basis of lesion area reduction and improvement of lesion scores. Although treatment efficacy was not formally evaluated after the Day 7 assessment, the qualitative improvement of the horses that continued treatment with the kunzea oil formulation, particularly the cases that crossed over from the control group after one (n=5) or four (n=1) weeks, supports the conclusion that the kunzea oil ointment was an effective treatment in this trial.

Pathological screening demonstrated staphylococcal involvement in most pastern dermatitis cases, with Staphylococcus aureus present in 15 cases and a caogulase-negative Staphylococcus sp. in three cases, two of these being mixed staphylococcal cultures. Only 4 from 20 cases screened did not culture a Staphylococcus sp. from a crust or hairmat sample. The major Gram-stain findings were of Gram-positive cocci, consistent with primarily staphylococcal infection, in 13 cases. Culture of samples from healed pastern dermatitis cases (when the lesion area was 0 cm²), including the two control cases that were cured, all resulted in negative staphylococcal and fungal culture. Gram-stain microscopic examination of smears also showed a reduction in the number of staphylococci, there being only two cases for which Gram-positive cocci were observed following treatment. It is apparent from these results that S. aureus played an important role in the pastern dermatitis cases of this study cohort—if not in the initiation of the underlying disease, then at least in the progression of the lesions. S. intermedius previously has been implicated in a case of vasculitis and systemic antibiotic therapy was effective in exacting a cure. It had been determined from previous studies that kunzea oil is potently bactericidal towards S. aureus and a range of other Gram-positive cocci and bacilli, as well as having activity against a range of yeasts and dermatophytes, including Trichophyton spp. and Microsporum canis. The potent activity of kunzea oil against staphylococci probably underlies its efficacy in the treatment of pastern dermatitis cases in this study. The antifungal activity of kunzea oil also contributes to its efficacy where fungi may play some part in the disease pathogenesis, although fungal involvement in the cases of the current study appeared to be less important than bacterial infection,

The Day 0 median value of the total lesion area in the control group was 81 cm² compared with 40 cm² in the test groups. These were not statistically different, and the lesion severity scores were very similar for the groups. Treatment outcome did not appear to relate to the initial lesion area. For example, the horse with the lesion having the second-greatest individual area (147 cm²) was completely healed after 7 days' treatment with the control formulation. There were some cases (2 from 10) that appeared to respond to treatment with the control formulation. These positive outcomes may have been due to some hitherto unsuspected anti-inflammatory, antibacterial and/or antifungal activity of the control formulation. This study was designed as a blinded study however the distinctive odour of kunzea oil made it possible for some horse owners to identify the kunzea oil formulation. This did not affect the assignment of medications, as the randomisation schedule was predetermined and medication jars were secured with a seal to prevent disclosure of the contents before admission of a horse to the study. Six veterinarians were involved in this study, introducing the potential for observer variability. However, each horse in the study was assessed by only one veterinarian, so that overall any variability would have been between cases and not within cases, thus limiting any impact on the statistical evaluation of the study results.

This study represents the first formal trial of a potential veterinary medication that demonstrates effectiveness in the treatment of pastern dermatitis. This composition can also be used effectively to treat other dermatological conditions in both humans and animals.

In conclusion, treatment of pastern dermatitis with a formulation containing kunzea oil, a potent antibacterial essential oil, cured most cases within 7 days and was more effective than the control formulation containing ketoconazole. The kunzea oil ointment was a safe, fast-acting and effective treatment for pastern dermatitis in the study cohort. The kunzea oil ointment can offer horse owners a convenient and effective treatment option for non-severe pastern dermatitis.

Thus, in summary this trial assessed the efficacy of an ointment containing kunzea oil for treatment of horses with localised acute or chronic pastern dermatitis. Horses (n=37) were randomly allocated to the treatment with an ointment containing either 20% kunzea oil (test) or 2% ketoconazole (control). The kunzea oil formulation resulted in a significant (P<0.01) decrease in total lesion area from a total lesion area of median value 40 cm² (range 3-252 cm²) to 0 cm² (range 0-34 cm²), with complete resolution of pastern dermatitis signs in 7 of 11 cases after 7 days. The control formulation resulted in no significant change in total lesion area, with complete resolution of pastern dermatitis signs in two of 10 cases after 7 days. The kunzea oil ointment was significantly more effective than control, based on both lesion area reduction and clinical scoring improvement (P<0.01). Horses treated with kunzea oil ointment beyond the 7 day period demonstrated improvement in their condition that was not formally assessed. Both ointments were well tolerated with no adverse reactions reported. The kunzea oil ointment was safe, fast-acting and effective in treating pastern dermatitis in the study cohort and may offer a suitable treatment option for pastern dermatitis in the general horse population.

### Example 2

A composition in accordance with one embodiment of the invention comprises the following ingredients:
Kunzea oil—10 to 40 weight %
Salicylic acid BP—2 to 10 weight %
Sulphur powder BP—2 to 10 weight %
Zinc oxide powder—1 to 7 weight %
Cod liver oil BP—10 to 15 weight %
Triethanolamine BP—10 to 20 weight %
Emulsifying ointment BP add to 100 gm.

This composition is appropriate for treating a range of skin conditions in animals, and is particularly useful for treating greasy heel in horses and similar animals.

### Example 3

A composition formed in accordance with another embodiment of the invention comprises the following ingredients:
Kunzea oil—15 to 20 weight %
Salicylic acid BP—5 to 7 weight %
Sulphur powder—7 to 10 weight %
Coal tar solution BP—12 to 15 weight %
Cod liver oil BP—7 to 10 weight %
Triethanolamine BP—7 to 10 weight %
Zinc oxide—3 to 5%
Emulsifying ointment BP add to 100 gm.

This composition is appropriate for treating a range of skin conditions in humans, and is particularly useful for treating psoriasis, and other dermatological conditions, including onychomycosis, or the like.

### Example 4

A composition formed in accordance with another embodiment of the invention comprises the following ingredients:

| | | |
|---|---|---|
| kunzea oil | 15 to 20 | weight % |
| coal tar solution | 5 to 10 | weight % |
| salicylic acid | 3 to 5 | weight % |
| isopropanol | 15 to 20 | weight % |
| tween 80 | 4 to 8 | weight % |
| glycerine | 10 to 20 | Weight % |
| cetrimide | 5 to 5 | weight % |
| vanillin | 1 to 2 | weight % |
| rose oil | 1 to 2 | weight % |
| ethanol | to 100 | weight % |

The first three items are the active ingredients of the composition in accordance with the present invention and the remainder form the base of the lotion for allowing application of the active ingredients to the affected area for treatment.

Such a composition is suitable for treatment for persons suffering from psoriasis, dermatitis, or similar complaints by forming the composition into a lotion and applying the lotion to the affected area, such as for example to the head to treat the infected area.

Reference to any prior art in the specification is not, and should not be taken as, an acknowledgment or any form of suggestion that this prior art forms part of the common general knowledge in Australia or any other country.

Many modifications may be made to the preferred embodiment of the present invention as described above without departing from the spirit and scope of the present invention.

It will be understood that the term “comprises” or its grammatical variants as used in this specification and claims is equivalent to the term “includes” and is not to be taken as excluding the presence of other features or elements.

TABLE 1
Baseline demographics of all the horses enrolled in the pastern dermatitis trial.

| | Age | Disease | | Pastern | | | Pastern(s) |
|---|---|---|---|---|---|---|---|
| Gender | (yrs) | duration | | colour | Breed | Housing | affected |
| gelding | 11-15 | 3 | months | white | thoroughbred | paddock | HR^{a} |
| mare | >15 | 8 | months | white | Arabian | paddock | FR^{b}, FL^{c}, HL^{d} |
| mare | >15 | 3 | months | white | Arabian | paddock | HL, HR |
| mare | 11-15 | 3 | months | white | Arabian | paddock | FR |
| gelding | ≦5 | 2 | weeks | white | thoroughbred | stabled | HR |
| mare | 6-10 | 1 | month | white | thoroughbred | stabled | HL |
| mare | >15 | 2 | days | black | American | paddock | HL |
| | | | | | quarter | | |
| gelding | ≦ | 2 | years | white | thoroughbred | stabled | HL |
| mare | 11-15 | 5 | years | brown | pony | paddock | HL |
| stallion | 11-15 | 2 | years | white | thoroughbred | paddock | HL |
| mare | >15 | 1 | month | white | thoroughbred | stabled | HL, HR |
| mare | 6-10 | 3 | weeks | white | Arabian | paddock | HR |
| mare | >15 | 2 | years | white | thoroughbred | paddock | HL, HR |
| | | | | | cross | | |
| mare | 6-10 | 4 | months | white | thoroughbred | paddock | FL, FR |
| | | | | | cross | | |
| gelding | ≦5 | 2 | weeks | white | Arabian | paddock | HR |
| mare | ≦5 | 4 | weeks | white | pony | paddock | FL |
| mare | ≦5 | 4 | weeks | white | pony | paddock | HR |
| gelding | ≦15 | NA^{e} | | white | thoroughbred | paddock | HR |
| | | | | | cross | | |
| mare | ≦15 | NA | | white | thoroughbred | paddock | FL |
| | | | | | cross | | |
| mare | >5 | 1 | month | white | Warmblood | paddock | FL, HR |
| mare | >15 | 5 | months | white | Arabian | paddock | FL, FR, HR, HL |
| mare | >15 | 7 | months | white | thoroughbred | paddock | HR |
| mare | >15 | 7 | months | white | thoroughbred | paddock | FL, FR, HR |
| gelding | >15 | 7 | months | white | Arabian | paddock | HL, HR |
| mare | 11-15 | 1 | month | white | Warmblood | paddock | FL, HL |
| mare | ≦5 | 1 | year | white | thoroughbred | stabled | HL |
| gelding | ≦5 | 1 | month | brown | Arabian | stabled | FL |
| gelding | ≦5 | 3 | weeks | white | thoroughbred | paddock | HL |
| mare | ≦5 | 3-4 | yrs | white | Arabian | paddock | HL |
| gelding | ≦5 | 2 | weeks | white | Arabian | paddock | HR |
| mare | 6-10 | 1 | month | white | thoroughbred | paddock | FL |
| gelding | 11-15 | 2 | months | white | thoroughbred | paddock | HR |
| mare | ≦5 | 1 | year | white | Arabian cross | stabled | HL |
| mare | 11-15 | 5 | months | white | Arabian cross | stabled | HL |
| mare | 11-15 | 2 | months | white | Arabian cross | stabled | FL, FR, HR, HL |
| gelding | ≦5 | 3 | years | white | Arabian cross | paddock | FL, FR, HR, HL |
| gelding | >5 | 1 | month | white | thoroughbred | paddock | HL |

^{a}hind right,
^{b}front right,
^{c}front left,
^{d}hind left,
^{e}data not available

TABLE 2
Comparison of pastern dermatitis lesion areas in cm² and lesion scores
(median and range) before and after 7 days of treatment with kunzea
oil ointment (test, N = 11) or control ointment (n = 10)

| | | | |
|---|---|---|---|
| | | | Wilcoxon signed-rank |
| Clinical parameter | D 0 | Day 7 | test* |

Total lesion score

| | | | | |
|---|---|---|---|---|
| Test | 7 (3-36) | 1 | (0-9) | <0.01 |
| Control | 7 (1-29) | 7 | (0-17) | NS^{b} |

| | | |
|---|---|---|
| Mann-Whitney test^{a} | NS^{b} | <0.05 |

Total lesion area

| | | | | |
|---|---|---|---|---|
| Test | 40 (3-252) | 0 | (0.34) | <0.01 |
| Control | 81 (4-264) | 70 | (0-143) | NS |

| | | |
|---|---|---|
| Mann-Whitney test^{a} | NS | <0.05 |

Target lesion score

| | | | | |
|---|---|---|---|---|
| Test | 4 (2-12) | 1 | (0-9) | <0.01 |
| Control | 4 (1-14) | 4 | (0-13) | NS |

| | | |
|---|---|---|
| Mann-Whitney test^{a} | NS | <0.05 |

Target lesion area

| | | | | |
|---|---|---|---|---|
| Test | 35 (3-207 | 0 | (0-34) | <0.01 |
| Control | 64 (4-147) | 39 | (0.143) | NS |

| | | |
|---|---|---|
| Mann-Whitney test^{a} | NS | <0.05 |

Highest lesion score

| | | | | |
|---|---|---|---|---|
| Test | 4 (2-12 | 1 | (0-9) | <0.01 |
| Control | 5 (1-14) | 4 | (1-3) | NS |

| | | |
|---|---|---|
| Mann-Whitney test^{a} | NS | <0.05 |

Highest lesion score

| | | | | |
|---|---|---|---|---|
| Test | 35 (3-207) | 0 | (0-34) | <0.01 |
| Control | 79 (4-147) | 39 | (0-143) | NS |

| | | |
|---|---|---|
| Mann-Whitney test^{a} | NS | <0.05 |

^{a}P-value,
^{b}not significant (P > 0.05)

TABLE 3
Summary of pathology results in kunzea oil ointment treated (test)
and control groups showing the number of horses with positive findings,
sampled before (Day 0) and after (follow up) treatment.

| | | | | |
|---|---|---|---|---|
| | Test | | Control | |
| | (n = 11) | Follow | (n = 9) | |
| Pathology test | Day 0 | up | Day 0 | Follow up |

Examination in smear

| | | | | |
|---|---|---|---|---|
| Gram + ve cocci | 7 | 2 | 5 | 4 |
| Gram + ve bacilli | 3 | 0 | 2 | 2 |
| Gram + ve bacilli | 1 | 0 | 0 | 2 |
| Yeasts | 0 | 0 | 0 | 0 |
| Leucocytes | 0 | 0 | 0 | 0 |

Bacterial culture

| | | | | |
|---|---|---|---|---|
| Staphyloccoccus spp. | 8^{a} | 0 | 8^{b} | 5^{c} |
| Dermatophilus congolensis | 0 | 0 | 0 | 0 |

Fungal culture

| | | | | |
|---|---|---|---|---|
| Microsporum equinum | 4 | 0 | 1 | 1 |
| Trichophyton spp. | 0 | 0 | 0 | 0 |
| Malasezzia pachydermatus | 0 | 0 | 0 | 0 |

^{a}^{⁵}pure S. aureus, 1 pure coagulase −ve Staphylococcus sp., 2 mixed cultures;
^{b}^{⁷}pure S. aureus, 1 pure coagulase −ve Staphylococcus sp.;
^{c}^{²}pure S. aureus, 3 mixed cultures

1. Composition for the treatment of a dermatological condition, said composition characterised in that the composition comprises a kunzea oil and/or a myrtaceous oil or a precursor of kunzea oil and/or myrtaceous oil, or a derivative of kunzea oil and/or myrtaceous oil, said composition being suitable for topical application to an area of skin that is affected by the dermatological condition over a range of temperatures, and wherein the viscosity modifier is incorporated into the composition for reducing temperature dependence of the viscosity of the composition to facilitate topical application of the composition to the affected area.

2. A method of treating a dermatological condition characterised in that the method comprises the steps of applying an effective amount of a therapeutic composition to a part of the body having the dermatological condition wherein the composition comprises a kunzea oil and/or a myrtaceous oil or a precursor of kunzea oil and/or myrtaceous oil, or a derivative of kunzea oil and/or myrtaceous oil and a viscosity modifier, said composition being suitable for topical application within a predetermined temperature range and wherein the viscosity modifier is provided for reducing temperature dependence of the viscosity of the composition thereby facilitating topical application of the composition to the affected area so that the composition remains where applied to a greater extent to provide an effective treatment.

3. A composition for treating dermatological conditions in animals, characterised in that the composition is suitable for topical application to an area of skin that is affected by a dermatological condition, the composition including kunzea oil and/or myrtaceous oil, or a precursor of kunzea oil and/or myrtaceous oil, or a derivative of kunzea oil and/or myrtaceous oil and a viscosity modifier wherein the viscosity modifier is for reducing temperature dependence of the viscosity of the composition wherein the kunzea oil or myrtaceous oil is obtained from a botanical plant.

4. The composition according to claim 1, characterised in that the composition includes salicylic acid, sulphur powder, cod liver oil and/or a homogenizing agent.

5. The composition according to claim 1, characterised in that the composition includes a coal tar solution.

6. The composition according to claim 1, characterised in that the viscosity modifier causes the composition to have a consistency suitable for topical application for temperatures in the range from about 0° C. to about 45° C., preferably in the range from about 5° C. to about 45° C., most preferably from about 10° C. to about 45° C.

7. The composition according to claim 1, characterised in that the viscosity modifier is zinc oxide.

8. The composition according to claim 7, characterised in that the zinc oxide is a powder.

9. The composition according to claim 1, characterised in that the therapeutic oil is an oil obtained from oils from Kunzea spp. including K. baxteri, K. ericoides, K. pomifera or K. ericifolia; oils from Melaleuca spp. including M. alternifolia or M. quinquinervia; oils from Leptospermum spp. including L. scoparium or L. petersonii; oils from Syzygium spp, including Z. aromaticum or Z. gardneri.

10. The composition according to claim 1, characterised in that the Kunzea oil contains sesquiterpenes.

11. The composition according to claim 1, characterised in that the Kunzea oil contains α-pinene, 1,8-cineole, α-terpineol, bicyclogermacrene, δ-cadinene, citronellol, calamenene, ledol, globulol, and/or viridflorol.

12. The composition according to claim 1, characterised in that the kunzea oil is extracted from Kunzea ambigua.

13. The composition according to claim 1 for treating psoriasis, seborrheic dermatitis, solar keratosis, fungal infections, greasy heel, dermatitis, onychomycosis, or similar conditions of the skin in both humans and animals.

14. A composition for treating dermatological conditions, the composition being suitable for topical application to an area of skin that is affected or infected by a dermatological condition, the composition comprising:
(i) Kunzea oil—10 to 40 weight %
(ii) Salicylic acid BP—2 to 10 weight %
(iii) Sulphur powder BP—2 to 10 weight %
(iv) Zinc oxide powder—1 to 7 weight %
(v) Cod liver oil BP—10 to 15 weight %
(vi) Triethanolamine BP—10 to 20 weight %
(vii) Emulsifying ointment BP add to 100 gm.

15. The composition according to claim 1, characterised in that the composition comprises:
(i) Kunzea oil—15 to 20 weight %
(ii) Salicylic acid BP—5 to 7 weight %
(iii) Sulphur powder—7 to 10 weight %
(iv) Coal tar solution BP—12 to 15 weight %
(v) Cod liver oil BP—7 to 10 weight %
(vi) Triethanolamine BP—7 to 10 weight %
(vii) Zinc oxide—3 to 5%
(viii) Emulsifying ointment BP add to 100 gm.

16. The composition according to claim 1 containing kunzea oil 10-40% by weight based on the total weight of the composition.

17. The composition according to claim 1 for treating dermatological conditions caused by bacteria cultured from Staphylococcus spp., Dermatophilus congolensis, Microsporum spp., Trichophyton spp., and or Malassezia pachydermatis.

18. A method of treating a dermatological condition, the method comprising applying an effective amount of composition according to claim 1, to an area of skin that is affected or infected by the dermatological condition, characterised in that the composition is applied twice a day to the affected area of skin.

19. The method according to claim 2 characterised in that the method is suitable for treating pastern dermatitis, greasy heel, psoriasis dermatitis, solar keratosis, onychomycosis, and/or fungal infections.

20. The method according to claim 2, characterised in that the method is for treating dermatological conditions in horses, cats, dogs, alpacas and other animals, preferably animals with unpigmented skin or white points.

21. The composition or according to claim 1 characterised in that the composition maintains an effective viscosity enabling the composition to be applied to a person or animal at an elevated temperature up to about 45° C., without significant loss of viscosity due to the elevated temperature.

22. The composition according to claim 1 characterised in that the viscosity modifier is zinc oxide having a particle size in the range from about nano-sized particles to micro-sized particles, preferably in the range less than 100 nm or in the range greater than about 100 nm, more preferably in the range of 0.01 μm to about 200 μm, even more preferably in the range of about 0.001 mm to about 0.002 mm.

23-24. (canceled)
